(19) United States
(12) Patent Application Publication
(10) Pub. No.: US 2016/0136284 A1
(43) Pub. Date: May 19, 2016

(54) GLIOMA TREATMENT

(71) Applicant: RENISHAW PLC, Gloucestershire (GB)

(72) Inventors: Steven Streatfield GILL, Bristol (GB); Edward WHITE, Bristol (GB)

(73) Assignee: RENISHAW PLC, Gloucestershire (GB)

(21) Appl. No.: 15/007,834

(22) Filed: Jan. 27, 2016

Related U.S. Application Data
(62) Division of application No. 14/379,358, filed on Aug. 18, 2014.
(60) Provisional application No. 61/609,631, filed on Mar. 12, 2012.

(30) Foreign Application Priority Data
Mar. 12, 2012 (GB) 1204263.6

(51) Int. Cl. A61K 47/46; A61K 31/282

(52) U.S. Cl. CPC A61K 47/46; A61K 31/282

(57) ABSTRACT

A method of treating a glioma is disclosed. The method includes administering to a patient in need thereof a composition that includes a chemotherapy agent and artificial cerebrospinal fluid via convection enhanced delivery.

This is a Divisional application of U.S. application Ser. No. 14/379,358 filed Aug. 18, 2014, which is in turn a National Stage application of International Application No. PCT/EP2013/055045 filed Mar. 12, 2013, which further claims priority from Application Nos. GB 1204263.6 and U.S. 61/609,631 filed on Mar. 12, 2012. The prior applications, including the specification, drawings and abstracts are incorporated herein by reference in their entirety.

### FIELD OF THE INVENTION

The invention relates to compositions, kits, and dosage regimens for treating brain tumours, especially gliomas.

### BACKGROUND TO THE INVENTION

Glioblastoma multiforme (GBM) is the most common and most aggressive form of primary brain tumour with an incidence of 2.8 cases per 100,000 per year in the United States. Despite extensive research the prognosis for patients with GBM remains bleak. Current treatment involves a combination of surgical resection, systemic chemotherapy and radiotherapy. However, due to the highly infiltrative nature of GBM and the intrinsic chemoresistance of GBM cells, 80% of tumours recur within 2 cm of the tumour resection cavity or in the context of tumours treated by radiotherapy and chemotherapy alone, recurrence most commonly occurs adjacent to the original tumour mass. As systemic dissemination of GBM is extremely rare and the median survival for recurrent GBM is typically less than 1 year, there is a clear and rational need for effective strategies aimed at improving local tumour control.

Techniques attempted in clinical trials to improve the local control of GBM have included the direct infusion or implantation of conventional chemotherapeutic agents such as carmustine, paclitaxel and topotecan, or novel cytotoxic agents, including oncolytic herpes simplex and adenoviral vector viral and non-viral mediated gene therapy and immunotoxins such as IL13-PE38QQR, into the tumour mass, resection cavity or peritumoural tissue. To date, the only technique of localised drug delivery that has become clinically accepted is the implantation of carmustine wafers (Gliadel) into the tumour resection cavity. However, a recent Cochrane Collaboration Review of the use of Gliadel wafers concluded that in combination with radiotherapy, Gliadel has survival benefits in the management of primary disease in a “limited number” of patients, but has “no demonstrable survival benefits in patients with recurrent disease”.

The principal limitation of many of the techniques of direct chemotherapy delivery to the brain, including Gliadel wafers, is their dependence on diffusion to achieve adequate spatial distribution within the brain. Diffusion is a highly inefficient process for drug distribution as it depends heavily on the infused drug concentration and molecular size of the drug. As a consequence, it is necessary to instill a very high concentration into the brain to generate an adequate concentration gradient which will distribute the drug a significant distance into the tissue.

For many chemotherapeutic agents, this source concentration is likely to be toxic to normal brain tissue, leading to significant side-effects. Convection-enhanced delivery (CED) offers an alternative strategy for infusing drugs into the brain. CED utilises implanted intracranial catheters through which drugs are infused at precisely controlled, slow infusion rates. The use of an appropriate catheter, trajectory and infusion rate leads to bulk flow of drug directly into the brain extracellular space.

In contrast to techniques of drug delivery to the brain that depend on diffusion, such as Gliadel wafers, which lead to heterogeneous drug distribution over short distances, depending on the size of the drug, CED is capable of distributing drugs, homogeneously, over large volumes of brain, independently of the size of the drug.

Whilst preclinical studies confirm that CED is a viable and potentially highly effective approach for administering drugs directly into the brain, it is not appropriate for all drugs. CED bypasses the tight junctions of the blood-brain barrier to allow drug distribution within the brain extracellular space. However, whilst highly lipophilic drugs, such as carmustine, may diffuse freely across the blood-brain barrier, other drugs such as paclitaxel may act as substrates to efflux transporters located within the blood-brain barrier, causing these drugs to be rapidly eliminated from the brain. It is therefore essential that in future trials utilising CED, therapeutic agents are carefully selected to ensure that they are retained in the brain for sufficient time for an anti-tumour effect to occur.

Carboplatin is a conventional chemotherapeutic agent that has been administered intravenously to patients with high-grade gliomas in isolation or in combination with erlotinib, tamoxifen, Gliadel, etoposide, human tumour-necrosis factor-α, thymidine, cyclophosphamide, RMP-7, ifosfamide and teniposide. Although these trials failed to demonstrate significant evidence of efficacy, carboplatin represents an excellent chemotherapeutic agent for administration by CED. It is a hydrophilic agent, ensuring that it is unable to diffuse freely across the blood-brain barrier and as such it is a substrate for the principal efflux transporters in the blood-brain barrier. As a consequence, direct intracranial administration of carboplatin by CED should result in drug compartmentalisation within the brain. There is also in vivo evidence, from infusions into animal models, demonstrating that carboplatin is highly efficient at killing glioblastoma cells at concentrations that are not toxic to normal brain tissue. Whilst some of these trials have had encouraging results, there is no convincing evidence that intravenous carboplatin administration confers significant benefit to patients with high-grade gliomas. However, there is compelling evidence that the concentration of carboplatin achieved within glioma tissue following intravenous administration is sub-therapeutic. Specifically, Whittle et al. demonstrated a peak glioma tissue concentration of just 0.013 mg/ml following high-dose intravenous delivery. Indeed this represents just 40% of the concentration that has been demonstrated, in a meta-analysis of published chemosensitivity assays, to kill 50% of tumour cells (IC₅₀) of carboplatin.

In view of the aforementioned data, the inventors realised that carboplatin administered at an appropriate concentration directly into the peritumoural region by CED has the potential to be an efficacious treatment for patients with GBM. This is in contrast to direct intratumoural infusions of carboplatin, which due to grossly abnormal tissue architecture, necrosis and neovascularisation within the tumour, is unlikely to be a practical approach. The inventors determined the tissue half-life of carboplatin administered by CED, and evaluated the distribution properties of carboplatin in both rat and pig brain, so as to the suitability of carboplatin for administration by CED. Additionally they assessed coinfusion of the MRI-contrast agent gadolinium-DTPA as a practical means for imaging carboplatin distribution clinically. As CED offers the possibility of producing sustained infusions of carboplatin over hours or even days, the inventors have evaluated the GBM tumour cell kill that can be achieved at a range of carboplatin concentrations and treatment durations in vitro. Finally, the inventors have undertaken a study to assess the toxicity of carboplatin administered by CED over a range of concentrations.

### SUMMARY OF THE INVENTION

The invention provides a pharmaceutical composition comprising chemotherapy agent and artificial cerebrospinal fluid (acsf). The inventors have found administering chemotherapy agent in conjunction with acsf to be particularly effective. Artificial cerebrospinal fluid as used in the present invention may comprise glucose, proteins and ionic constituents. In preferred embodiments of the invention the artificial cerebrospinal fluid does not comprise glucose or proteins.

The composition preferably comprises the chemotherapy agent at a concentration of between 0.01 mg/ml and 0.30 mg/ml, more preferably at a concentration of at least 0.02 mg/ml, 0.03 mg/ml, 0.06 mg/ml, 0.09 mg/ml, 0.12 mg/ml, 0.15 mg/ml, or 0.18 mg/ml, and/or more preferably at a concentration of less than 0.27 mg/ml, 0.24 mg/ml, 0.21 mg/ml, 0.18 mg/ml, 0.15 mg/ml, 0.12 mg/ml, or 0.09 mg/ml. In embodiments of the invention the composition may comprise a chemotherapy agent at a concentration of between 0.01 mg/ml and 0.7 mg/ml, preferably between 0.02 mg/ml and 0.6 mg/ml, most preferably between 0.03 and 0.5 mg/ml.

Also provided is chemotherapy agent, or a composition according to the invention, for use in the treatment of brain cancer, wherein the chemotherapy agent is for administration by convection enhanced delivery. Alternatively, there is provided a chemotherapy agent for use in the preparation of a medicament for the treatment of brain cancer, wherein the agent is for administration by convection enhanced delivery.

Convection enhanced delivery is well known in the art. It means the delivery of a pharmaceutical, or other composition, to the brain by a narrow catheter, usually having an inner diameter of less than 500 μm, more usually less than 250 μm.

The chemotherapy agent is preferably for administration via at least one convection enhanced delivery catheter, especially an intraparenchymal catheter. More preferably it is for delivery via at least two, at least three or four or more such catheters. Preferably the catheter or catheters are for implantation into white matter, particularly such that the distal end of the catheter, from which the infusate exits the catheter is in white matter, such as white matter within 5, 10, 15, 20, 25 or 30 mm of a glioma or of a site from which a glioma has been resected. Alternatively the catheter may be for implantation with its distal end in a tumour.

One or more catheters may be chronically implanted into a patient allowing repeat infusions of the chemotherapy agent.

The chemotherapy agent is preferably for administration at a concentration of between 0.01 mg/ml and 0.30 mg/ml, more preferably at a concentration of at least 0.02 mg/ml, 0.03 mg/ml, 0.06 mg/ml, 0.09 mg/ml, 0.12 mg/ml, 0.15 mg/ml, or 0.18 mg/ml, and/or more preferably at a concentration of less than 0.27 mg/ml, 0.24 mg/ml, 0.21 mg/ml, 0.18 mg/ml, 0.15 mg/ml, 0.12 mg/ml, or 0.09 mg/ml. In embodiments of the invention the chemotherapy agent may be for administration at a concentration of less than 1 mg/ml, 0.9 mg/ml, 0.8 mg/ml, 0.7 mg/ml, 0.6 mg/ml, 0.5 mg/ml, 0.4 mg/ml or 0.3 mg/ml. In a further embodiment of the invention the chemotherapy agent may be for administration at a concentration of 0.72 mg/ml or less.

The chemotherapy agent is preferably for administration by infusion for between 4 and 24 hours, especially for at least 5, 6, 7, 8, 9, 10, 11, 12, 13, 14, 15 or 16 hours and/or for less than 23, 22, 21, 20, 19, 18, 17, 16, 15, 14, 13, 12, 11, 10, 9 or 8 hours. It is preferably for infusion for around 8 hours. In embodiments of the invention the chemotherapy agent is for infusion over a period of at least 48 hours, preferably at least 72 hours.

The chemotherapy agent is preferably for administration on at least two, preferably three, optionally four consecutive days. Alternatively, the chemotherapy agent may be for administration on two out of three, four or five days, or three out of four, five, six or seven days.

Whether or not the chemotherapy agent is for administration for a number of consecutive days or for regular administration over a number of days, it may independently or additionally be for administration weekly, fortnightly, monthly, every six, eight, twelve or fifteen or more weeks. For example, a cycle of two or three days of infusions may be repeated every fortnight. Alternatively, it may be for administration in a series of cycles of infusions, with 6, 7, 8, 9, 10, 11 or 12 days between the end of a first cycle of infusions and the next cycle of infusions.

For example, the chemotherapy agent may be for administration by infusion for between 6 and 10, especially between 7 and 9 hours, each day for three consecutive days. This pattern of administration may then be repeated weekly, or fortnightly, or for example with 6, 7, 8, 9, 10, 11 or 12 days between the end of a first cycle of three days of infusions and the next three days of infusions.

The chemotherapy agent is preferably for administration at a flow rate of at least 6 μl/min, more preferably at least 7, 8, 9, 10, 11, or 12 μl/min, and/or at a flow rate of less than 15, 14, 13, 12, 11, 10, or 9 μl/min. In embodiments of the invention the chemotherapy agent is infused at rates of 20 μl/min or less, preferably 15 μl/min or less, more preferably 10 μl/min or less.

The chemotherapy agent is preferably for administration at a rate of at least 10 μl/min in a 24 hour period, more preferably at least 12, 14, 16, 18, 20, 22, 24, 26, 28, or 30 μl/min, and/or less than 40, 38, 36, 34, 32, 30, 28, 26, 24, 22, 20, 18, 16 μl/min in any 24 hour period.

The chemotherapy agent may be for administration after treatment of a glioma, such as by resection or by radiotherapy. It may also be for administration prior to or after administration of a different therapeutic agent, especially another chemotherapy agent.

Also provided is a method of treating a glioma comprising administering a chemotherapy agent via convection enhanced delivery. The chemotherapy agent may be administered using any of the administration route, methods, dosages, rates etc., mentioned above.

In particular there is provided a method for treating a glioma comprising implanting a convection enhanced delivery catheter having proximal and distal ends, such that its distal end is implanted in white matter within 5, 10, 15, 20, 25 or 30 mm of a glioma or of a site from which a glioma has been resected, and delivering a chemotherapy agent via the catheter. In an embodiment of the invention the chemotherapy agent may delivered into a tumour and its penumbra. Generally the penumbra incorporates a margin of at least 20 mm around the tumour as visualised by Magnetic Resonance Imaging (MRI).

The chemotherapy agent may be delivered at any of the dosages, concentrations or flow rates etc described herein.

The method preferably comprises implanting more than one, especially two, three or four catheters, preferably all with their distal end in white matter within 5, 10, 15, 20, 25 or 30 mm of a glioma or of a site from which a glioma has been resected.

Preferably the method includes delivering a chemotherapy agent by infusion for around 6, 8, 10 or 12 hours. More preferably the method includes delivering a chemotherapy agent by infusion for up to 24 hours.

In a preferred embodiment of the invention the method allows a sustained therapeutic dose of the chemotherapy agent to be delivered for at least 48 hours. In further embodiments of the invention the sustained therapeutic dose is maintained for at least 72 hours.

The method preferably comprises delivering the chemotherapy by infusion on two, three or four consecutive days.

The method preferably comprises delivering the chemotherapy agent at a concentration of between 0.03 and 0.18 mg/ml, more preferably between 0.03 and 0.36 mg/ml.

Further provided is a kit for treating a glioma comprising at least one catheter having an internal diameter of less than 500 μm, and a dose of a chemotherapy agent arranged to deliver the chemotherapy agent at a concentration or flow rate or for an infusion time as described above. The kit may comprise two, three, four or more catheters. Appropriate catheters are described in WO03/077785. It may also comprise a port for connecting the catheters to a delivery device. Such ports are described in WO2008/062173 and WO2011/098769.

Also provided is a dosage vessel comprising a chemotherapy agent, wherein the dosage vessel is arranged to deliver the chemotherapy agent at a concentration or flow rate or for an infusion time as described above. The dosage vessel may be for example a sealed tube that can be connected in fluid communication to a port as described.

The chemotherapy agent may be any chemotherapy agent suitable for treating tumours, especially a cytotoxic agent. In particular, it is preferably a hydrophilic chemotherapy agent, especially one which cannot cross the blood brain barrier. It is preferably carboplatin, cisplatin, oxaliplatin, topotecan, doxorubicin, paclitaxel or gemcitabine, especially carboplatin.

The cancer may be any cancer of the brain or upper spinal cord, especially a glioma. It may be a primary cancer or a metastasis from a cancer outside the brain. The tumour may be a tumour that is not amenable to surgical resection, such as a tumour of the brainstem.

The invention will now be described in detail, by way of example only, with reference to the drawings.

### DESCRIPTION OF THE DRAWINGS

FIG. 1: Laser Ablation Inductively Coupled Plasma Mass Spectrometry (LA-ICP-MS) Following In Vivo Infusions

Rats were infused with 0.03 mg/ml carboplatin into the corpus callsosum. This is represented in the relevant coronal image from the Paxinos and Watson rat brain atlas (top left). The cannula position is demonstrated by the location of the arrow. Rat brains were harvested at time-points of 0, 3, 6, 12, 24, 48, 72 hours and 7 days. Coronal sections at the level of the cannula track were analysed by LA-ICP-MS. Results at each time point (n=3) are shown in images a-h. The top row of images for each time-point show tissue maps of platinum levels (¹⁹⁵Pl) and the lower row of each image show tissue maps of iron levels in each section (⁵⁷Fe).

FIG. 2: Time-Course of Tissue Levels of Platinum Following Carboplatin Infusions

Graphical representation of the average (a) and maximum (b) amount of platinum (ng of platinum/g of tissue) detected by LA-ICP-MS on all three sections analysed at each time point following carboplatin infusions into rat brain.

FIG. 3: Carboplatin Toxicity In Vivo

Rat brains were infused with 0.9 mg/ml (a), 0.6 mg/ml (b), 0.3 mg/ml (c), and 0.03 mg/ml (d) of carboplatin. Tissue sections were evaluated at 24, 48 and 72 hours and 30 days to assess for evidence of tissue toxicity. Control infusions of 0.9% saline were performed and tissue analysed at 24 hours (e) and 30 days (f). Representative images of haematoxylin and eosin staining (left column), GFAP immunostaining (middle column) and the myelin staining (DiI-right column) are shown. No evidence of tissue toxicity was observed at any carboplatin dose compared to controls (scale bar=500 μm).

FIG. 4: Dose Response of Glioma Cell Lines to Carboplatin In Vitro

(a) UPAB (b) SNB19 glioma cells were exposed to increasing carboplatin concentrations for 24, 48, 72 and 96 hours. Cell viability was assessed by MTA (graphs show values for group mean (n=3) and standard deviation). With a 96 hour exposure, there was a negligible increase in cell kill (greater than 90%) at concentrations exceeding 0.18 mg/ml. Similarly, with a 72 hour exposure, there was a negligible increase in cell kill at concentrations exceeding 0.24 mg/ml.

FIG. 5: Comparison of T1-Weighted MR Imaging and LA-ICP-MS Following Coinfusion of Carboplatin and Gadolinium-DTPA into the Corona Radiata of a Pig

Carboplatin (0.03 mg/ml) was coinfused with gadolinium-DTPA (0.3%; (6 mmol/l) into the corona radiata of a pig bilaterally (a-c: right hemisphere, d-f: left hemisphere). T1-weighted MR images (a and d) and LA-ICP-MS images of ¹⁵⁷Gd (b and e) and ¹⁹⁵Pl (c and f) distribution on corresponding tissue sections are shown. T1-weighted MR imaging demonstrated a close correlation between contrast-enhancement and carboplatin distribution. LA-ICP-MS was more sensitive than T1-weighted MR scanning at visualising gadolinium distribution and demonstrated that gadolinium-DTPA distributed over a larger volume of brain than carboplatin. (All scale bars=5 mm).

FIG. 6: Carboplatin Toxicity

The toxicity of carboplatin was analysed by measuring the levels of synaptophysin in the brain. A significant reduction in synaptophysin is indicative of pre-synaptic toxicity and was observed at 0.72 mg/ml carboplatin but not at 0.36 mg/ml.

FIG. 7: Preoperative Planning—Left Transfrontal Trajectory for Catheter Implantation

Trajectory (a), sagittal (b), axial (c) and multi-planar views (d) of the tumour using an in-house modification to Neuro|Inspire™ stereotactic planning software. This software facilitated analysis of tumour volume and planning of a left transfrontal catheter trajectory.

FIG. 8: Preoperative Planning—Manufacture of a Bespoke Catheter with a Winged Hub

Pre-operative trajectory planning facilitated the in-house manufacture of a bespoke catheter composed of PEEK bonded onto fused silica and with a winged hub section (a). In-house software was used to output stereotactic co-ordinates to the Neuro|Mate® Robot Used for guide-tube and catheter implantation (b). On implantation of the catheter, a 3 mm section of fused silica was retained within the distal end of the guide-tube thus creating a recessed-step (c). A diagrammatic representation of the externalised catheter tubing and in-line gas and bacterial filter on the head is shown (d).

FIG. 9: Infusions of Carboplatin—T2 Weighted MRI Scan for Volumetric Analysis of Signal Change as a Proxy Measure of the Final Infusate Distribution

Axial (a) and multi-planar (b) T2-weighted MR images on completion of the final infusion. Hyperintense signal change was used as a measure of infusate distribution within the tumour (shown in green). The volume of T2 signal change represented 95% of the targeted tumour volume.

FIG. 10: Clinical and Radiological Follow-Up—T2 Weighted MRI Scan

Comparison of T2-weighted MR imaging prior to treatment (a, c & e) and corresponding images at 4 weeks post infusion (b, d & f) revealed areas of increased hyperintensity within the left cerebral peduncle, as well as within the mid and lower pons suggestive of the early stages of tumour necrosis. However, there was evidence of continued tumour progression at the inferior and anterior aspects of the tumour, which were outside the volume of T2 signal change on cessation of the infusions.

### EXAMPLE 1

### Methods/Design

### Study Design:

This is a phase I, single centre, dose-escalation study, of carboplatin administered by CED, into the peritumoural region of patients with recurrent or progressive GBM, following tumour resection. The study will incorporate six cohorts, with three patients in each cohort. Patients will be recruited sequentially to each cohort and the infusion concentration of carboplatin increased from one cohort to the next, subject to dose-limiting toxicity not occurring. The trial will be conducted at Frenchay Hospital (North Bristol NHS Trust, Bristol, UK).

### Treatment Intervention

The treatment plan is shown in Table 1. Following tumour resection (study day 0), patients undergo a baseline MRI scan (study day 10-21), followed within 24 hours by catheter implantation. For patients with progressive multifocal disease in whom re-resection is not felt to be appropriate, catheters are implanted following a baseline MRI scan. Prior to catheter implantation, patients are loaded with phenytoin (unless allergic) and will be kept on this anticonvulsant during the duration of their infusions. In the event of allergy to phenytoin, patients are administered an alternative anticonvulsant during this time period. Catheters are stereotactically implanted in the vicinity of the resection cavity with a view to distributing carboplatin within 3 cm of the resection cavity. Catheter tip location is determined at the operating surgeon's discretion, and will be based on diffusion imaging-based fibre-tracking using iPlan Flow (Brainlab, Germany) and experience gained in animal models and previous patients. Up to four catheters are implanted per patient under general anaesthetic and attached to connector tubing, which will be tunnelled to a subcutaneous access device incorporating in-line bacterial filters, implanted in the infraclavicular fossa. Four small drug infusion ports are connected transcutaneously to this subcutaneous access device. Following catheter insertion an MRI scan is performed to confirm that each catheter has been inserted accurately to target. Should there be misplacement of a catheter it is removed and replaced with a new catheter.

Once patients have recovered from catheter implantation and their wounds have healed, ward-based infusions of carboplatin is undertaken. The first infusion is undertaken 21 to 28 days after catheter implantation. Infusions are performed for 8 hours a day for 3 consecutive days. Patients undergo MR imaging before and after each daily infusion to evaluate carboplatin distribution. The infusion rate employed does not exceed 10 μl/min per catheter and no more than 20 ml of infusate is infused per day. If patients develop a headache or neurological deficit during infusions, the flow-rate may be reduced to 50/min and the infusion duration prolonged for up to 16 hours.

Patients undergo up to four sets of infusions of carboplatin during the course of the trial. Each set of infusions consists of 8 hour infusions conducted on consecutive days for 3 days. The interval between sets of carboplatin infusions is between 4 and 7 days. For each infusion, patients are admitted to hospital and have external syringes/tubing connected to the drug infusion ports and carboplatin infusions undertaken on the ward.

The patients in each cohort receive the same treatment, but each cohort receives increasing concentrations of carboplatin (Table 2). Patients are recruited to the cohort receiving the lowest drug concentration initially. If treatment is completed without evidence of significant toxicity, the next cohort is recruited to receive a higher drug concentration. Should a patient in a treatment cohort develop life-threatening toxicity, or if 2 out of the 3 patients develop Eastern Cooperative Oncology Group (ECOG) grade 3 or 4 toxicity then all patients in that cohort have their drug concentration changed to the concentration below and no further dose-escalation is undertaken. There is at least a 28 day delay before carboplatin is administered to patients in the next cohort to ensure that any relevant toxicity is detected. Interim data analysis is performed by the trial monitoring committee after all patients have been treated in each cohort and after one month has elapsed to determine whether the next cohort should be treated at a lower dose, the same dose or the next dose in the dose-escalation regime. This dose escalation strategy facilitates calculation of the maximum tolerated infusion concentration (MTIC).

Following completion of their last infusion, patients are followed up in clinic at 1 month, 2 months and then 3-monthly with an MRI on the same day. At their 1-month follow-up appointment, patients are weaned off their phenytoin (or other anticonvulsant), if they have not had seizures at any time.

The inventors have realised that carboplatin administered by CED prolongs survival in animal models of high-grade glioma, even if the tumour is located in the brainstem. Furthermore, the only published primate study to date demonstrates that despite carboplatin infusions lasting up to one month, very low serum carboplatin levels are observed. In the context of intravenous administration in humans, peak carboplatin concentrations have been reported to lie in the range of 84 to 140 μmol/L representing serum concentrations more than 5 thousand times more concentrated than the 6 μg/L observed following intracranial infusions of 0.25 mg/kg of carboplatin in primates. As a consequence it is unsurprising that none of these studies led to significant systemic toxicity and in particular myelosuppression, which is the major dose-limiting toxic effect of intravenously administered carboplatin. However the majority of monkeys did lose weight presumably due to stimulation of the chemoreceptor trigger zone in the brainstem resulting in chronic nausea.

This clinical trial incorporates 4 sets of 3 infusions of carboplatin. Each infusion lasts 8 hours. Infusions are performed on consecutive days for 3 days and then 4 days later the next set of infusions will begin. Consequently patients will undergo 12 infusions over a 28 day period. This represents a safer dosing strategy for treating patients than undertaking a single prolonged infusion over one month (as conducted by Strege et al, 2004 in primates), particularly as little is known about the pharmacokinetics of carboplatin, or any other drug infused directly into the brain. However, it is likely that as the extracellular fluid turnover of the brain is between 10 and 17 hours, that most unbound drug will have diffused or been effluxed across the blood-brain barrier, or washed into the subarachnoid space within a few days, limiting further tumour cell kill. Furthermore, this infusion strategy is more practical than the infusion strategy employed by Strege et al (2004). Specifically, it minimises the hospital stay of patients and the use of higher flow-rates has the advantage of maximising carboplatin distribution through the brain, limiting the risk of toxic concentrations being achieved at the catheter tip. Nevertheless the most obvious parallel between animal studies and this clinical trial is the prolonged dosing regime employed by Strege et al (2004).

Extrapolating the work of Strege et al (2004) would suggest that the lowest concentration of drug that would be likely to lead to significant toxicity would be 0.22 mg/ml (assuming a patient weight of 70 kg). By undertaking infusions intermittently, allowing tissue recovery, the inventors have been able to increase the concentration used. Carboplatin distribution is achieved over a larger volume of brain due to the use of much higher flow-rates (10 μl/min vs. 0.42 μl/hr).

The toxicity observed by Strege et al, which manifested in the form of ataxia and lethargy is likely to relate to local brain dysfunction. In the context of infusing carboplatin into cerebral tumours, such toxicity is unlikely to be so pronounced.

In view of these differences, it seems likely that the maximum-tolerated dose in humans may be significantly higher than 0.22 mg/ml using the inclusion/exclusion criteria and delivery strategy outlined.

It should be noted that cohort 1 receives a carboplatin concentration equivalent to the IC₅₀ of carboplatin in relation to glioblastoma cells in vitro. Indeed this IC₅₀ value assumes a 96 hour exposure of tumour cells to the chemotherapeutic agent being examined By undertaking infusions on 3 consecutive days the inventors maintain this carboplatin concentration within the vicinity of tumour cells for 96 hours, essentially replicating a similar tumour cell kill. Indeed, for patients in which a 50% tumour cell kill can be achieved with each carboplatin infusion (at 0.03 mg/ml), then 4 consecutive sets of infusions should lead to a theoretical 94% tumour cell kill at this lowest dose.

### Implantable Catheter System

The drug delivery catheter systems used in this study are in-house devices as used in the inventors long-term infusion study of glial cell line-derived neurotrophic factor (GDNF) in patients with Parkinson's disease that had continuous intraparenchymal infusions to the striatum for up to 4 years Gill, S. S et al (2003), Patel et al (2005).

Each system comprises 4 intraparenchymal catheters that are connected by extension tubing, tunnelled subcutaneously to an access device containing in-line bacterial and bubble filters. The access device is approximately the size of a cardiac pacemaker and is implanted in the subclavicular region. Four small externalised infusion ports are connected to the access device by fine tubes that pass transcutaneously to connectors on the access device. The infusion ports are 12 mm×6 mm cylinders with a proximal septum seal and a distal winged hub from which extends the fine connection tubing (0.55 mm diameter). Each winged hub is sutured to the skin immediately proximal to the site of skin penetration to fix the ports for the duration of the trial and minimise movement of the connection tubing through the skin.

Infusions are commenced by placing 4 carboplatin filled syringes into pre-programmed syringe drivers (B. Braun Medical Ltd, UK) to each of which is attached connection tubing, an in-line bacterial and bubble filter and a butterfly needle. Under asepetic conditions the ports are cleaned with alcohol, which is allowed to dry and then further cleaned with sterile saline. The butterfly needles are inserted into each port and secured to the anterior chest wall with adhesive tape for duration of the infusion (8 to 16 hours). Between infusions the infusion lines will be disconnected and the infusion ports protected with a light dressing.

The externalised ports remain in-situ for the duration of the trial or for the life of the patient if they so wish. They will be removed if they become infected or at the patient's request. This will most probably be performed under a short general anaesthetic. The remainder of the drug delivery system will not be removed unless it is deemed necessary, for example due to infection, as this would expose patients to unacceptable risk.

### Patient Monitoring

Monitoring of patients during the trial are conducted as follows:
Regular neurological observations are performed before, during and after carboplatin infusions. The frequency of these observations is tailored according to clinical experience with carboplatin infusions. These include hourly assessments of the patient's vital signs and Glasgow Coma Score during the infusion and 4-hourly observations after infusion completion.
Full patient assessment against ECOG criteria daily during carboplatin infusions.
Daily full blood count, urea, creatinine, electrolytes and liver function tests during hospital admission and prior to starting each carboplatin infusion.
Immediate cessation of infusion +/−MRI scan if NEW or WORSENING focal neurology identified or Grade 4 ECOG toxicity identified.
If patients develop symptoms of potential toxicity to the treatment intervention between infusions or with 1 month of infusion completion, patient's is clinically assessed by a member of the study team and a decision will be made whether the patient's clinical features are attributable to the treatment intervention, and if they are whether they constitute ECOG grade 3 or 4 toxicity. If the patient is deemed to be demonstrating grade 4 toxicity, all further infusions in the study are performed at the next lowest dose. If grade 3 toxicity is observed, no further infusions will be undertaken until all available outcome data is evaluated to determine whether other patients in that treatment cohort have also demonstrated grade 3 toxicity. If this is the case, all further infusions in the study are performed at the next lowest dose.
MRI scans daily during carboplatin infusions to ensure adequate catheter perfusion volume and distribution.
MRI scans at 3 month intervals to monitor tumour progression.
European Organisation for Research and Treatment of Cancer (EORTC) Validated patient quality of life questionnaire before and after all infusions and at all follow-up assessments (EORTC QLQ-C30 with brain module).
Duration of patient survival.

### Study End-Points

### Primary Outcome Measures:

Maximum tolerated infusion concentration (MTIC).
Complications/side-effects/tolerability/toxicity (ECOG criteria) of treatment.

### Secondary Outcome Measures:

Serial quality of life measurements at 3-month intervals.
Progression-free survival (PFS) based on serial MRI scans at 3-month intervals.
Overall survival.
Relationship between catheter location and visible carboplatin distribution based on MRI.
Relationship between carboplatin distribution, PFS and overall survival.
Serum carboplatin pharmacokinetics during/after intracranial infusions.

### Definition of Study End

As patients will be followed up for a maximum of 2 years, this study will end 2 years after the last infusion has been completed. Alternatively, if it occurs sooner, the study will end when all the patients have died or are withdrawn from the study at the request of the patient or their oncologist.

### Data Analysis

Data analysis will performed on patients on an intention-to-treat basis. This will include:
Toxicity within each treatment cohort using the Eastern Cooperative Oncology Group toxicity grading scale.
Kaplan-Meier survival analysis will be performed.
A qualititative assessment of the optimum MR sequences for visualising infusate distribution.
Volumetric analysis of carboplatin distribution from MR images.

### Discussion

In view of the in vitro sensitivity of glioblastoma cells to carboplatin at concentrations that appear not to be toxic to normal brain in vivo, the inventors have realised that carboplatin administered at an appropriate concentration directly into the peritumoural region by CED has the potential to be an efficacious treatment for patients with GBM. The key element to this is to achieve effective and widespread carboplatin distribution by CED. Indeed it is obvious that drug distribution and drug efficacy are inextricably linked. However, achieving effective drug distribution by CED, in contrast to most other techniques of drug administration, depends on numerous variables that can be modulated easily by clinicians. These include catheter tip location, catheter trajectory, catheter design, infusion volume and infusion flow-rate. Changing these variables can dramatically effect drug distribution through the brain.

When CED has been employed in previous large-scale clinical trials to administer a variety of immunotoxins to patients with high-grade gliomas, drug distribution was generally suboptimal and this is likely to have contributed to the failure of these trials to demonstrate evidence of drug efficacy. For example, when drug distribution was evaluated in the phase III PRECISE immunotoxin trial using coinfused radiolabelled HSA and SPECT imaging, 50% of catheters demonstrated no significant infusate distribution in the brain despite catheters initially being considered to be optimally positioned (Sampson et al, 2007).

Careful examination of preclinical data evaluating the importance of catheter outer diameter and infusion flow-rate on achieving CED, would suggest that drug distribution in these immunotoxin trials would be poor. Specifically Chen et al (2005) demonstrated that to achieve CED the catheter outer diameter should be significantly less than 1 mm. Indeed this study demonstrates that the use of a catheter with an excessive outer diameter leads to extensive reflux along the catheter/brain interface. In contrast the PRECISE trial employed a 2 mm outer diameter catheter and an infusion flow-rate of 12.5 μl/min. Similar catheters were used in numerous clinical trials treating patients with malignant gliomas, administering the immunotoxins TP-38 (personal communication with IVAX) and transferrin-CRM107, as well as paclitaxel.

There are a number of reasons why inappropriate catheters have been used in clinical trials. First and foremost, there are no commercially available catheter systems that are compatible with CED, and consequently the inventors have developed an appropriate catheter in-house. There are a number of technical reasons as to why the development of such catheters is challenging. Firstly, it is difficult to develop catheters with a diameter of significantly less than 1 mm that can accurately be inserted over many centimetres into the brain without bending off target or breaking. Indeed two approaches have been developed to deliver catheters into the brain, including the use of a rigid guide-wire inserted through the centre of the cannula to make it sufficiently rigid to insert to target. However, upon removing the guide-wire and initiating the infusion, a column of air (within the catheter) is infused directly into the brain causing a cavity to be formed at the catheter tip, limiting drug distribution. Alternatively, catheters can be delivered through a rigid guide-tube. However, this acts to create a low-resistance pathway around the outside of the catheter along which reflux occurs. The catheter system that the inventors have developed incorporates a series of implantable guide-tubes that overcomes these problems and facilitates the accurate implantation of catheters over large distances of brain. Indeed this catheter incorporates a stepped outer surface that has been shown to facilitate high flow CED with minimal reflux.

In the earlier study using CED to administer glial cell line-derived neurotrophic factor (GDNF) into the putamen of patients with Parkinson's disease, the inventors used an identical 0.6 mm outer diameter catheter and an infusion flow-rate of 0.1 μl/min. In contrast, this study requires effective drug distribution to be achieved over a much larger volume of brain. As a consequence, up to four catheters are inserted and infusion flow-rates of at least 5 μl/min are employed. Whilst this flow-rate has the potential to cause extensive reflux, the highly anisotropic nature of the white matter will facilitate effective carboplatin distribution. Furthermore, the inventors have experimental evidence that chronic catheter implantation minimises infusate reflux. Indeed, the inventors have tested the performance of a 0.6 mm outer diameter catheter in a large animal model and have demonstrated that it is possible to achieve effective infusate distribution without demonstrable reflux at flow-rates of 5 μl/min. Previous studies have employed the software application iPlan Flow (Brainlab, Germany) to predict infusate distribution for specific catheter locations and trajectories. This has allowed surgeons to optimally place catheters relative to the target volume. However, this software remains unvalidated and the use of a similar patient-specific algorithm for predicting infusate distribution by CED has been shown to be unreliable. As a consequence catheter tip placement in this study is based on a combination of software predictions and empirical findings.

Finally, carboplatin represents an ideal therapeutic agent for direct intracranial administration to treat malignant brain tumours. The hydrophilic nature of carboplatin ensures that whereas intravascular administration leads to sub-therapeutic drug concentrations in the brain, this property ensures that carboplatin administered directly into the brain should be compartmentalised by the blood-brain barrier, limiting the risks of systemic toxicity.

### EXAMPLE 2

### Materials and Methods

### In Vitro Studies

### Cell Lines and Cell Culture:

Cell lines were kindly provided by Geoffrey Pilkington from the Institute of Biomedical and Biomolecular Sciences at Portsmouth University, UK. The cell lines used in this study were SNB19 (P20-P45) and UPAB (P20-P45).

### MTT Cytotoxicity Assay:

Briefly, SNB19 and UPAB glioma cells were plated at 1×104/well in a 24-well plate. Cells were treated 72 hours later with Carboplatin at the following concentrations: 0.06 mg, 0.12 mg, 0.18 mg, 0.24 mg, 0.3 mg, 0.36 mg, and 0.6 mg (TEVA, UK) for 24, 48, 72, or 96 hours. Each concentration was repeated 4 times. Carboplatin was diluted in phosphate buffered saline (PBS; Sigma Aldrich, UK) and added to 0.5 mL culture media. PBS was used as a negative control and Puromycin dihydrochloride (10 μg/mL; Sigma Aldrich, UK), which inhibits cell growth by preventing protein synthesis, was used as a positive control. Following the incubation period, culture media was changed. Then 50 μL Methylthiazolyl-tetrazolium bromide solution (MTT; Sigma Aldrich, UK; 5 mg/mL) was added to each well and further incubated for 3 hours at 37° C. in a humidified 5% CO2 atmosphere to allow MTT to form formazan crystals in metabolically active cells. Following this, media was removed, and the formazan crystals in each well were solubilized with 190 μL of isopropanol (Fisher Scientific, Loughborough, UK) acidified with hydrochloric acid (VWR, Leicestershire, UK). The cell lysate was transferred to a 96 well plate and the absorbance of each well was measured at 570 nm using a Multiskan Ascent plate reader (Thermo Electron Corporation, UK). Results are expressed as a percentage (%) of treated versus untreated cells.

### In Vivo Studies

### Rat Infusion Apparatus and Procedures:

All acute infusions were undertaken using in-house cannulae White et al (2011) and all procedures were carried out in accordance with UK Home Office animal welfare regulations and with appropriate Home Office licenses.

Male Wistar rats (Charles River, UK) were group-housed and allowed to acclimatise prior to experimental procedures. Rats weighing 225 to 275 g were anaesthetised with an intraperitoneal dose of medetomidine (Dormitor; 0.4 mg/kg; Pfizer Animal Health, Kent, UK) and ketamine (Ketaset; 100 mg/kg; Pfizer, UK) and placed in a stereotactic frame (Stoelting Co, Wood Dale, Ill. USA). A linear incision was made between the glabella and the occiput and the skull exposed. Burr holes with a diameter of approximately 2 mm were drilled 1.0 mm anterior and 2.5 mm lateral to the bregma and cannulae were inserted to a depth of 2.5 mm below the dura. All cannulae were pre-primed with either saline or carboplatin and the desired dose prior to insertion into the brain. Every attempt was made to ensure that no air bubbles were present in the infusion cannula. Infusions of 2.5 μl of carboplatin at specific concentrations (outlined in Table 1) were conducted at a rate of 5 μl/min. Following infusion completion, the cannula was left in-situ for 10 min before being withdrawn at a rate of 1 mm/min. The wound was then closed with 5.0 Vicryl (Ethicon, Gargrave, UK) a dose of intramuscular buprenorphine was administered (Vetergesic; 0.03 mg/kg; Alstoe Animal Health, York, UK) and the anaesthetic was reversed with an intraperitoneal dose of atipamezole hydrochloride (Antisedan; 5 mg/kg; Pfizer, UK). At predetermined time-points (see Table 1), animals were perfusion fixed with 100 mL of PBS followed by 100 mL of 4% paraformaldehyde (PFA; Fisher Scientific, UK) in PBS (pH 7.4). The brain was then removed from the skull and placed in 4% PFA for 48 hours and then cryoprotected in 30% sucrose (Melford Laboratories, Ipswich, UK) in PBS prior to sectioning.

### Pig Infusion Apparatus and Procedures:

Carboplatin infusions were undertaken into male Large White Landrace pigs weighing 45 kg using a cannula system developed in-house. Pig anaesthesia, head immobilisation and brain imaging were achieved as we have previously described White et al (2010). Infusions of 120 μl 0.03 mg/ml carboplatin mixed with 0.3% (6 μmol/l) Gadolinium-DTPA (Magnevist: Bayer Healthcare, Germany), were undertaken bilaterally into the corona radiata using a cannula composed of a length of fused silica (outer diameter 220 μm, inner diameter 150 μm) bonded to a glass Hamilton syringe. Except for the distal 3 mm, this fused silica tube was supported along its length by a series of zirconia tubes to ensure that it could be accurately inserted to target. Infusions were performed using the following regime: 0.5 μl/min for 5 min, 1 μl/min for 5 min, 2.5 μl/min for 5 min and then 5 μl/min for 20 min. This regime was employed in an attempt to minimise the occurrence of a sudden surge in pressure at the catheter tip due to elasticity in the infusion tubing. 120 μl was infused as this is the largest volume that we have previously infused into pig white matter without leakage into the ventricular system. Following infusion completion, the cannula was left in place for 10 min prior to being withdrawn slowly by hand. CSF leakage from the burr hole and cannula track was sealed with Cerebond prior to wound closure. The animal was then transferred back to the MRI scanner and T1-weighted imaging performed to visualise infusate distribution. Upon the completion of imaging, the animal was transcardially perfused with 5 L of PBS and then 5 L of 10% neutral-buffered formalin at a rate of 500 ml/min using an infusion pump (Mastedlex, UK).

### Histology

Rat brains were cut into 35 μm thick coronal sections using a Leica CM1850 cryostat (Leica Microsystems, Wetzlar, Germany) at −20° C. For haematoxylin and eosin staining, fixed sections were mounted on gelatine-subbed slides. Sections were submerged in 4% PFA for 20 minutes, dehydrated and then stained with haematoxylin and eosin (Cell Path, Hemel Hempstead, UK) according to standard protocols. Following this, sections were coverslipped with Pertex mounting medium (Cell Path, UK) and allowed to dry in the fumehood overnight before imaging with a Leica CTR 5500 microscope (Leica Microsystems, Germany). Sections were assessed by light microscopy to ensure that the cannula track in each brain terminated in the corpus callosum. If the cannula track did not terminate in the corpus callosum, the brain was excluded from further analysis and the infusion was repeated.

For fluorescent immunohistochemistry, free-floating sections were washed with PBS for 5 min×3. Sections were then blocked in PBS plus 0.1% triton-x-100 (Sigma Aldrich, UK) containing 10% normal donkey serum (Sigma Aldrich, UK) for 1 hour at RT. Sections were then washed with PBS for 5 min. Following washing, sections were incubated in polyclonal rabbit Anti-Glial Fibrillary Acidic Protein primary antibody (GFAP; 1:300; Millipore, Watford, UK) at 4° C. overnight. The next day, primary antibody was removed and sections were washed with PBS for 15 min×3. Secondary antibody (donkey anti-rabbit Cy3 1:300; Jackson Laboratories, Sacramento, Calif., USA) was added to the sections and incubated at RT for 1 hour in the dark and then washed with PBS for 15 min×3. Sections were mounted in Fluorsave mountant (Calbiochem, Germany) before viewing and image capture with a fluorescent microscope (Leica Microsystems, Germany) and digital camera (CX9000 Microbrightfield, VT, USA).

For DiI staining, free-floating sections were washed with PBS for 5 minutes. Sections were then submerged in a solution of 4′,6-diamidino-2-phenylindole (DAPI, 0.25 mg/ml, Sigma Aldrich, Gillingham, UK) in PBS for 5 minutes. After three PBS washes, sections were mounted onto gelatine-coated slides and stained with FAST-DiI oil (0.25 mg/ml; Invitrogen, Paisley, UK) diluted in 1:3 N,N,N′,N′-Tetramethylethylenediamine (TEMED, Sigma Aldrich, UK) and ddH₂O for 2 minutes. Slides were washed with ddH₂O and coverslipped using Fluorsave mountant. Once dry, slides were imaged with a fluorescent microscope (Leica Microsystems, Germany) and digital camera (CX9000 Microbrightfield, VT, USA).

### Laser Ablation Inductively Coupled Plasma Mass Spectrometry:

Samples were placed in a sealed ablation chamber under an Argon gas flow. Laser interrogation caused sample vaporisation; ablated material was then transported from the sample cell to the inductively coupled plasma (ICP) torch via an argon gas flow. Upon reaching the ICP the sample was completely atomised and ionised via high temperature plasma (7500-10000 K). Ions were then focused through a series of sampling cones and ion-lenses before isotopic mass discrimination (via quadrupole) for elements of interest and subsequent detection of ions (as electron multiplier (EM) detector counts).

Resultant data (.csv files) was in the form of signal response for each monitored isotope (separate columns) against time; as such, ion-responses could be co-ordinated to form 2D elemental distribution maps, using the Graphis software package (Kylebank Software Ltd, Ayr, UK).

The laser ablation (LA) system was configured to perform multiple, parallel line-rastering of sections. Operating parameters ensured efficient removal of sample (i.e. total consumption of thin section incident to the laser) irrespective of section thickness. Additionally, a distance twice that of the laser beam diameter was used to separate raster lines, to prevent contamination of adjacent section areas with ejected material from previous raster runs. Main operating parameters for ICP-MS (HP 4500, Agilent Technologies, Cheadle, UK), were: ICP forward power, 1340 W; plasma gas flow, 16 ml/min and auxiliary flow, 1.0 ml/min. Isotopes (¹³C, ⁵⁷Fe, ⁶⁶Zn, ¹⁵⁷Gd and ¹⁹⁵Pt) were monitored in a time-resolved mode and selected on the basis of high-percentage abundance and minimal isobaric and polyatomic interferences. Integration times for isotopes were 0.1 s (0.05 s for ¹³C).

### Rat Brain Analysis:

The laser ablation system (New Wave UP MACRO, Nd:YAG, 266 nm) was configured to the following parameters: beam diameter, 240 μm; laser energy, 2.2 mJ; line raster rate, 50 sec⁻¹; laser frequency, 10 Hz. A check standard (0.2 μg g⁻¹) was ablated at the beginning and end of each section interrogation in order to verify system stability. Total runtime for mapping individual sections (area 140-160 mm²) was approximately 2 hr 30 min.

Matrix-matched standards were prepared as previously described, at corresponding thickness to brain sections and contained known amounts of Pt at 0.01, 0.1 and 0.2 μg g⁻¹ (plus a blank). Standards were placed adjacent to the samples in the ablation chamber and triplicate line rasters (2 mm in length) performed prior to and after brain section analysis, on each standard. LA-ICP-MS conditions were identical to those used for tissue section analysis. Average ¹⁹⁵Pt ion-responses of individual rasters were plotted against spiked concentration to yield linear calibration graphs of the form y=mx+c. This permitted distribution maps to be displayed in concentration units.

For determination of average and maximum Pt concentrations, data was processed (using MS Excel) such that all on-tissue Pt signal responses above background levels were included; with the omission of Pt signals in areas co-localising with high-intensity Fe signals. These areas were consistent with small haemorrhages caused by cannula insertion and generally resulted in anomalously high Pt response, likely due to Pt capture in haemorrhagic components.

### Pig Brain Analysis:

The laser ablation system (Cetac, LSX-200, Nd:YAG, 266 nm) was configured to the following parameters: beam diameter, 100 μm; laser energy, 0.99 mJ; line raster rate, 65 μm s⁻¹; laser frequency, 10 Hz. Total runtime for mapping individual sections (scanned section areas were in the region of 30 mm by 30 mm) was approximately 12 hr.

### Results

Tissue Distribution and Half-Life of Carboplatin Following CED into Rat Brain:

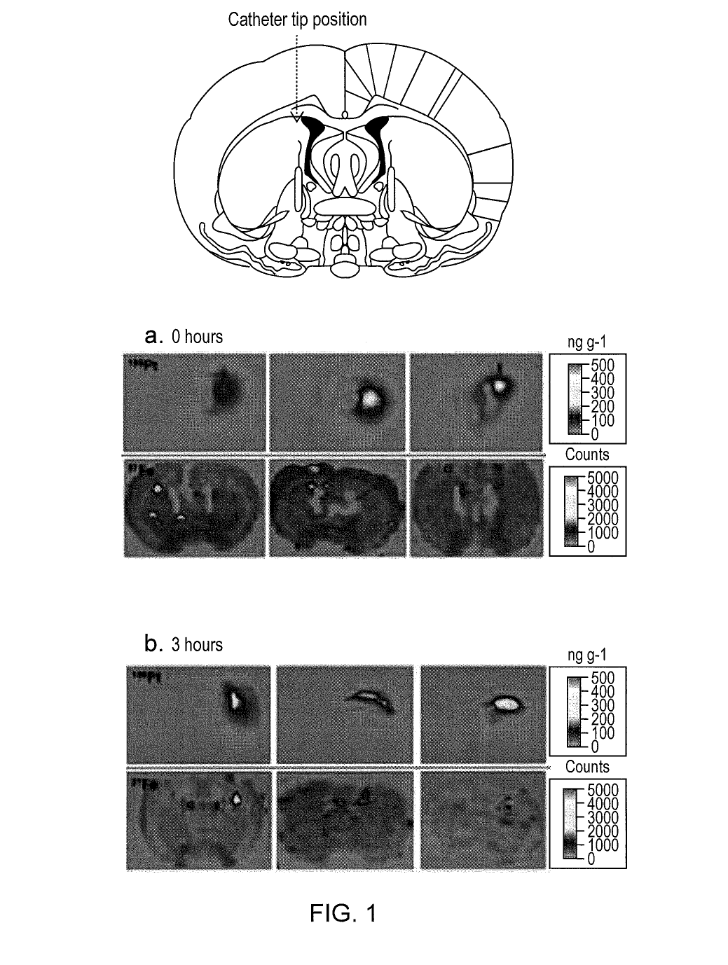

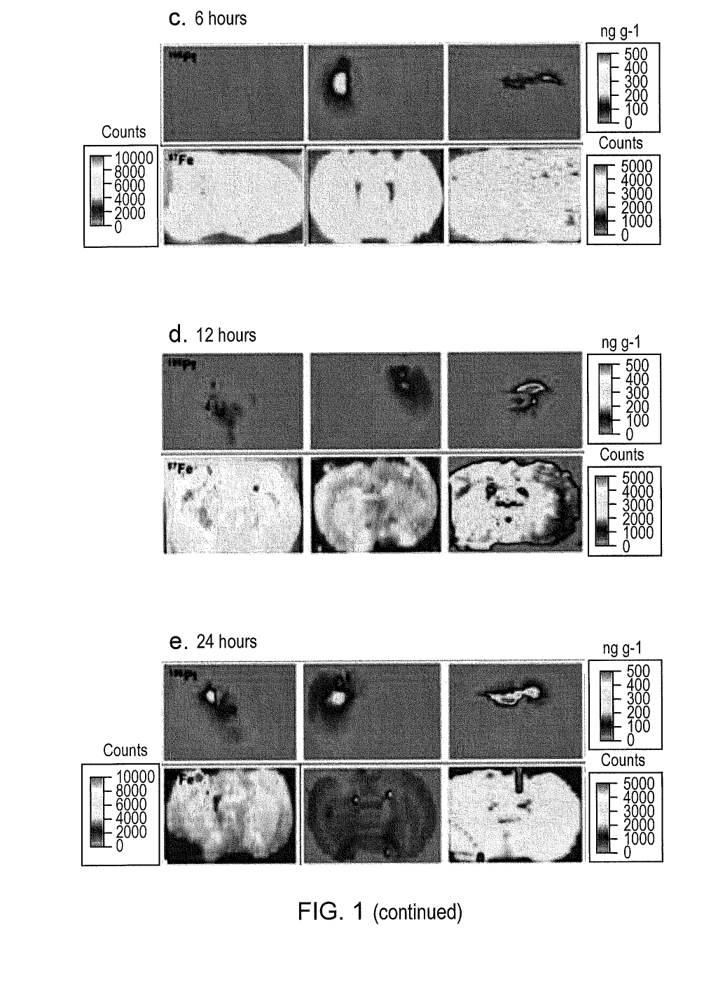

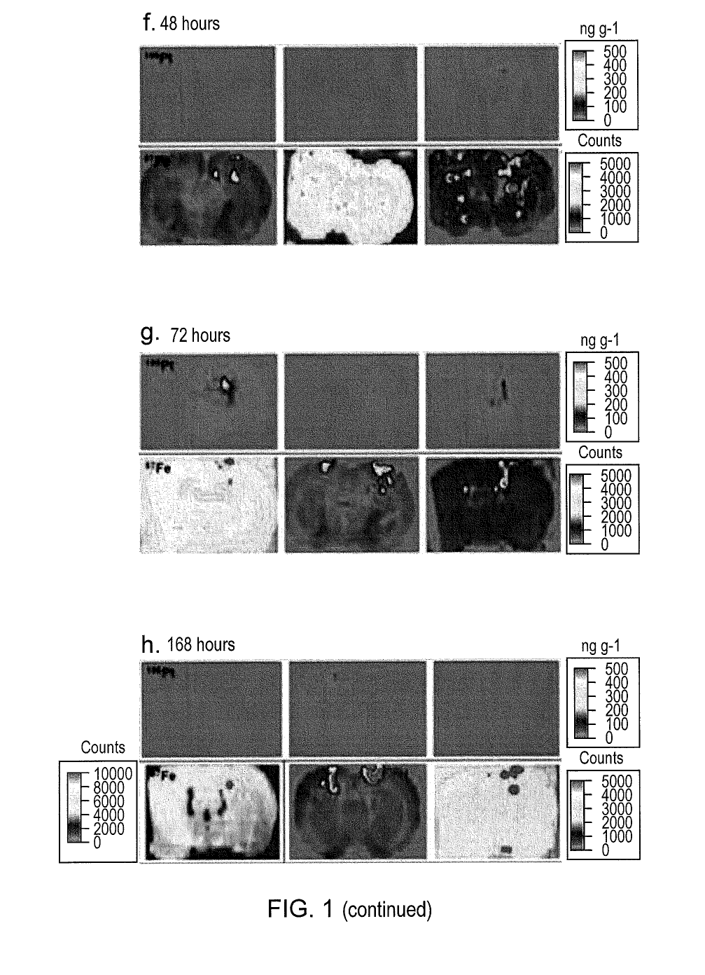

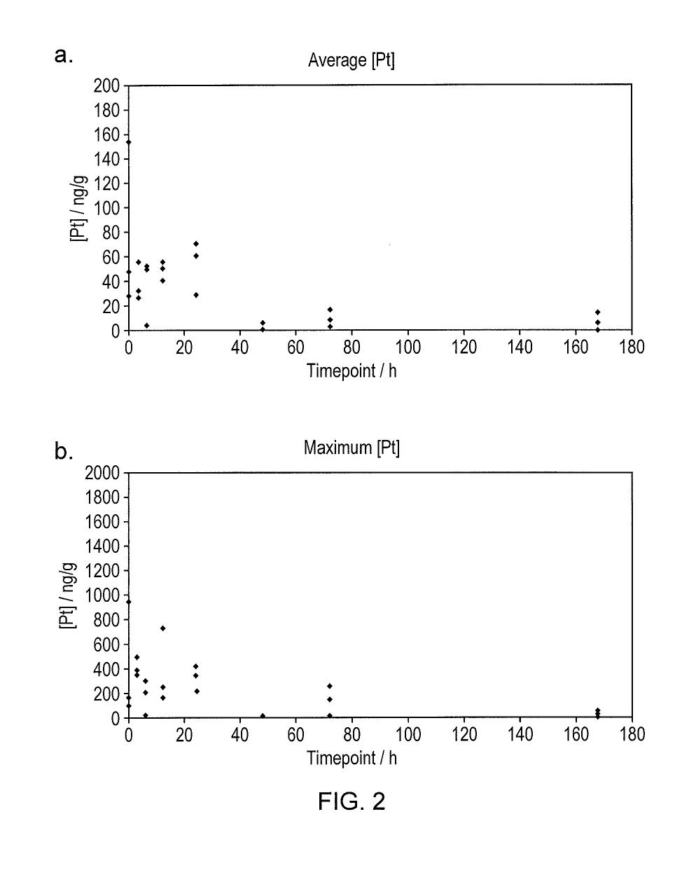

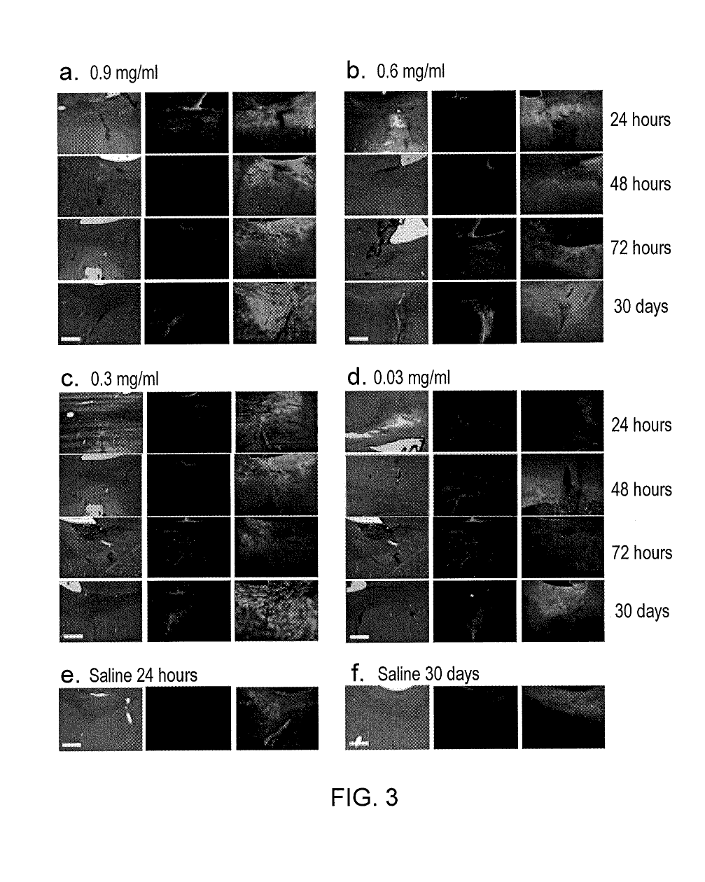

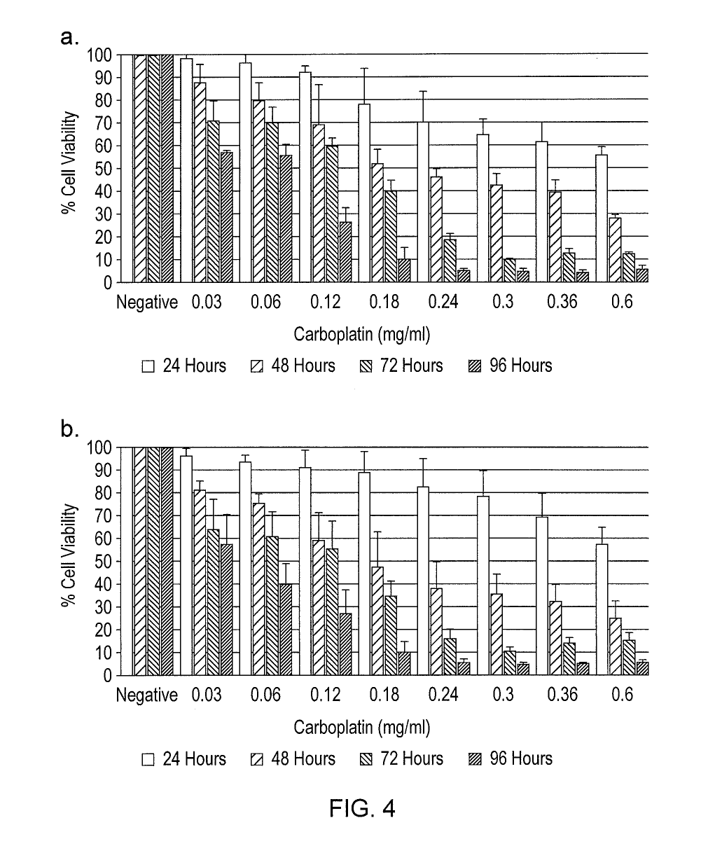

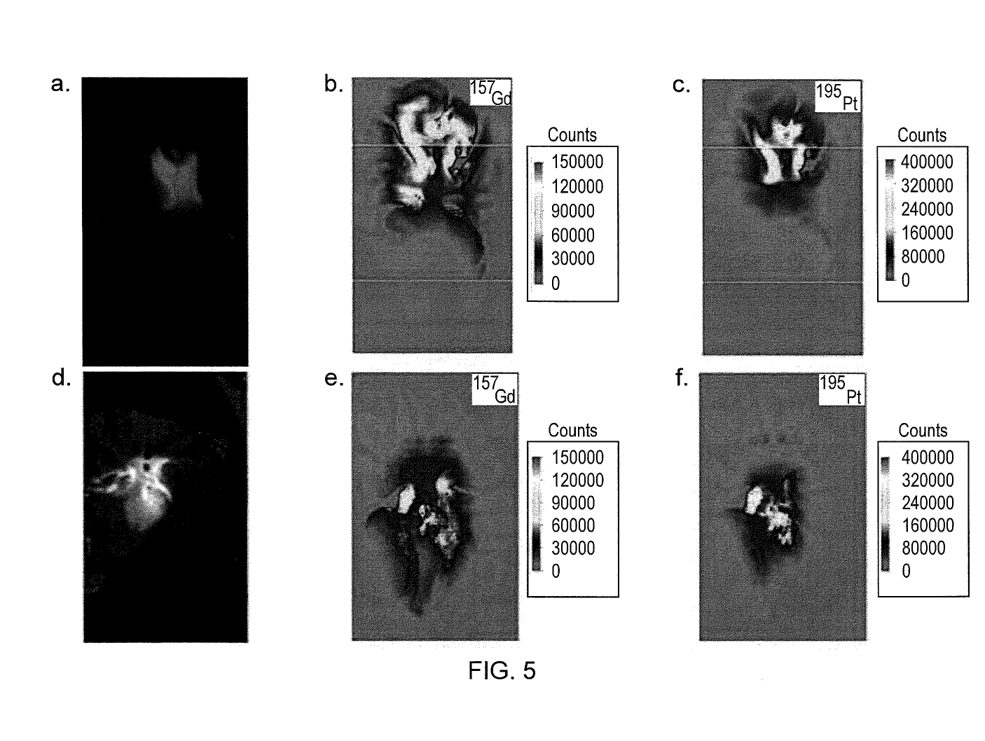

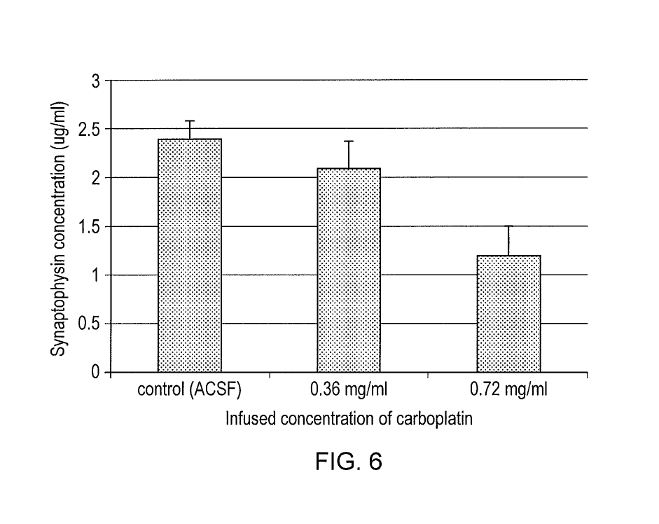

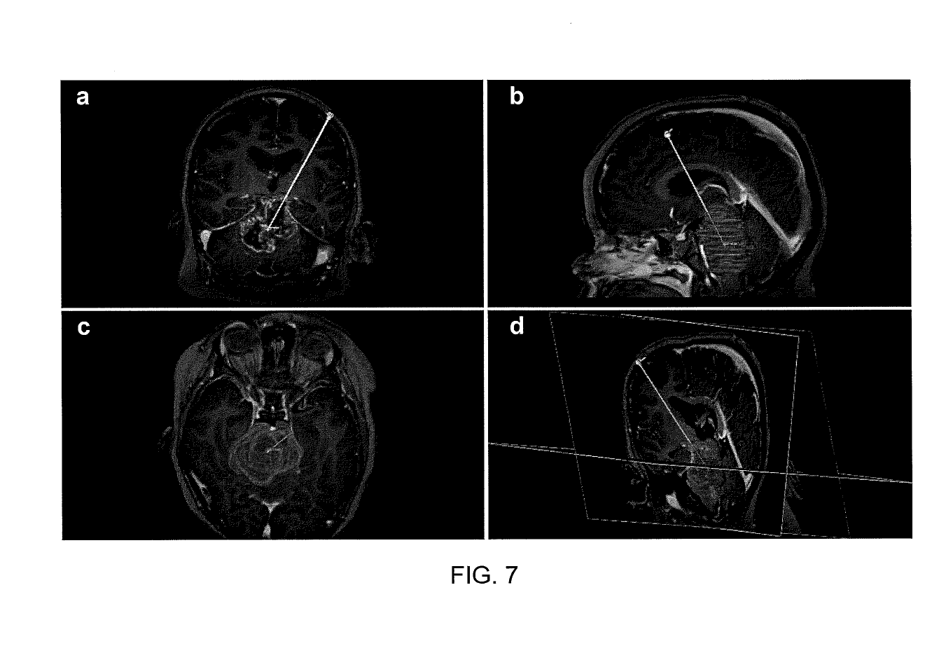

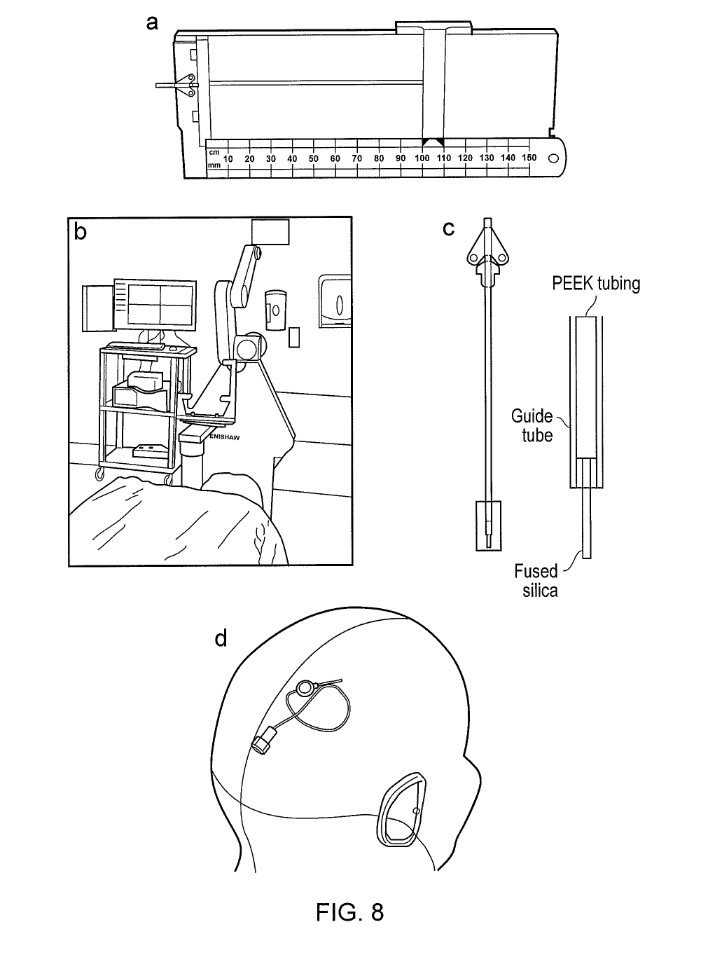

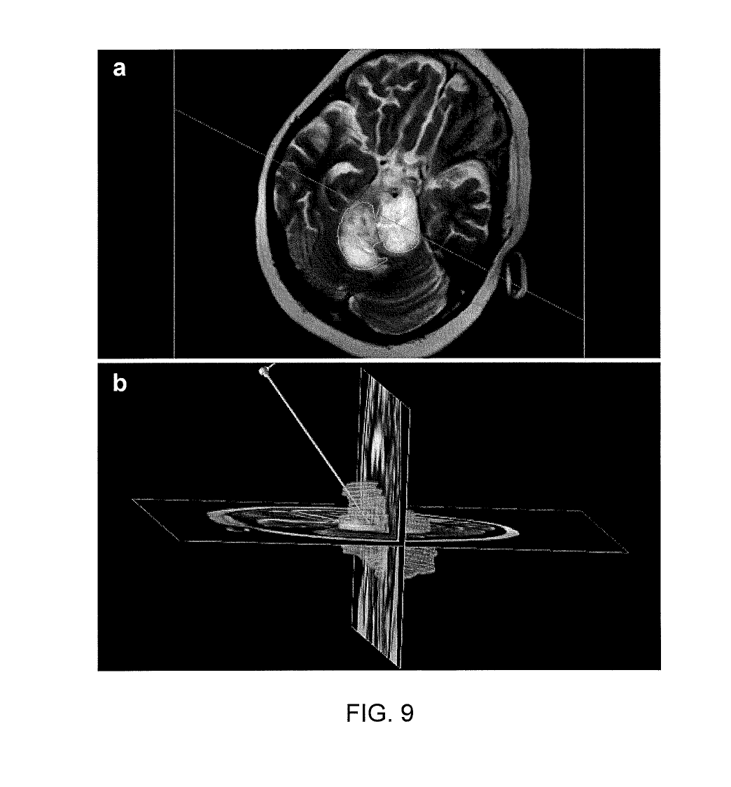

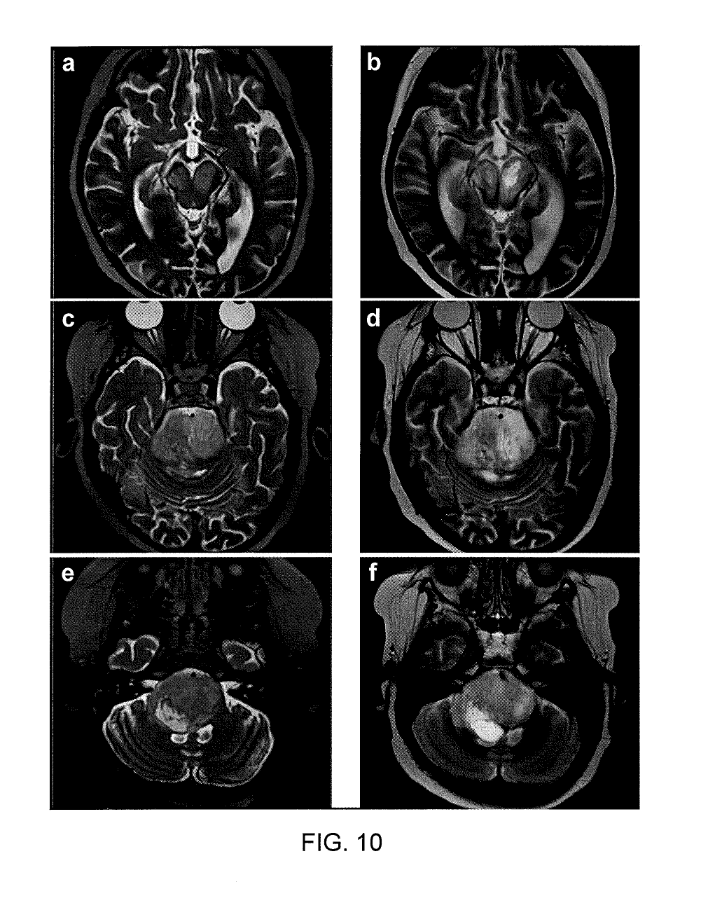

Low concentration infusions of carboplatin (0.03 mg/ml) into rat brain led to widespread distribution at 0 hours post-infusion. Although all infusions were performed through cannulae implanted into identical coordinates in the corpus callosum as defined by the Paxinos and Watson Sereotactic Rat Brain Atlas (1998), variable distribution patterns were observed. Apart from a single infusion analysed at 6 hours, platinum was detectable by LA-ICP-MS for up to 24 hours. After 24 hours, trace levels of platinum were detected in a number of tissue sections. In these sections platinum, colocalised with high levels of iron derived from small haemorrhages along the cannula tracks (FIG. 1). The decrease in carboplatin concentration over time was reflected in measures of average and maximum platinum counts for each section at each time-point (FIG. 2).

### Carboplatin Toxicity in Rat Brain:

Increasing concentrations of carboplatin were infused into the corpus callosum of rats. Histological examination of brains was undertaken 30 days post-infusion. Concentrations of up to 0.9 mg/ml were well tolerated no clinical evidence of toxicity and no histological evidence of tissue disruption based on haematoxylin and eosin staining (FIG. 3). Furthermore, DiI staining demonstrated no loss of white matter tract integrity and GFAP immunostaining showed minimal evidence of gliosis in the white matter compared to control infusions of 0.9% saline.

### In Vitro Dose Response to Carboplatin in Glioblastoma Cell Lines:

MTT assays in glioblastoma cell lines exposed to carboplatin at different concentrations for increasing durations demonstrated a clear relationship between carboplatin concentration and duration of carboplatin exposure on the percentage of surviving cells compared to controls (FIG. 4). With a 96 hour exposure, there was a negligible increase in cell kill (greater than 90%) at concentrations exceeding 0.18 mg/ml. Similarly, with a 72 hour exposure, there was a negligible increase in cell kill at concentrations exceeding 0.24 mg/ml. Based on these results the IC₅₀ value of carboplatin, assuming a 96 hour exposure of carboplatin, was between 0.06 and 012 mg/ml.

### Gadolinium-DTPA Coinfusion to Visualise Carboplatin Distribution by MRI:

Gadolinium-DTPA (0.3%; 6 μmol/l) was coinfused with 0.03 mg/ml of carboplatin into the corona radiata of pigs. T1-weighted MR imaging demonstrated a close correlation between contrast-enhancement and carboplatin distribution. LA-ICP-MS was more sensitive than T1-weighted MR scanning at visualising gadolinium distribution and demonstrated that gadolinium-DTPA distributed over a larger volume of brain than carboplatin although widespread carboplatin distribution was observed through the corona radiata (FIG. 5).

### Discussion

In view of the highly infiltrative properties of malignant gliomas and their subsequent propensity to recur adjacent to tumour resection margins, the rarity of extracranial disease dissemination and the grim prognosis associated with this disease, there is a clear and rationale need to improve local tumour control. This requirement is complicated by the presence of the blood-brain barrier, which limits the access of chemotherapeutic agents into the brain, tumour infiltration into eloquent structures and the intrinsic chemo- and radioresistance of glioblastoma cells. The principal aim of the experiments outlined in this study was to determine whether carboplatin, administered by convection-enhanced delivery into peritumoural brain, is a potentially feasible treatment to achieve local control of glioblastoma multiforme. Specifically, these experiments demonstrate that it is possible to achieve widespread carboplatin distribution by CED and that carboplatin remains in the brain for at least 24 hours. Furthermore, we provide evidence from in vitro studies that carboplatin is capable of killing a significant proportion of GBM cells at concentrations that appear to be well-tolerated in the brain in vivo. Finally we demonstrate that coinfusion of gadolinium-DTPA with carboplatin and peri-infusional T1-weighted MRI represents a viable technique for visualising carboplatin distribution in clinical practice. The results of this study have informed the development of a phase I/II clinical trial protocol that we intend to enact in the near-future.

CED of carboplatin into the corpus callosum of rats led to surprisingly variable distribution patterns. Two main patterns were observed with many infusions preferentially distributing through the striatum rather than the corpus callosum. This is likely to have occurred as the corpus callosum is a very shallow structure in rats and subtle variations in cannula tip position, despite using identical stereotactic coordinates, would have led to variable distribution patterns. In particular, if the cannula tip had been implanted fractionally too deep, carboplatin may have distributed into the striatum rather than along the corpus callosum. Attempts to ensure consistent cannula tip targeting in this study included the use of identical stereotactic coordinates for cannula insertion, the use of rats with an identical weight and examining tissue sections prior to undertaking LA-ICP-MS, to ensure that the cannula track was visible and terminated in the corpus callosum. However, in view of the very narrow cannulae employed in these infusions to achieve CED and the fact that brains were not harvested for up to 1 week, it was challenging to identify the cannula trajectory in many cases and this may explain why one 6 hour time-point demonstrated no detectable carboplatin. Furthermore there is intrinsic variability with tissue analysis by LA-ICP-MS although regular machine calibration ensured that this was less than 15%. Despite these potential sources of variability, carboplatin was visible in tissue sections that demonstrated preferential distribution in the striatum and in the corpus callosum (FIG. 1e) for at least 24 hours. The observation of low levels of carboplatin at time points beyond 48 hours and the approximate colocalisation of platinum with areas of iron, most likely reflects binding of carboplatin to serum proteins or haemoglobin at the site of trivial haemorrhages along the cannula track.

The presence of significant concentrations of carboplatin at 24 hours is an encouraging finding and supports our hypothesis that due to its hydrophilic nature and subsequent inability to diffuse freely across the blood-brain barrier, carboplatin is an ideal agent to be delivered directly into peritumoural brain. Indeed, these findings are supported by similar clearance times calculated for radiolabelled albumin following injection into the caudate nucleus and internal capsule of rats. Consequently, this relatively prolonged tissue half-life ensures that carboplatin can be distributed over large volumes of brain, despite the low flow-rates that are demanded by CED. Furthermore, this relative compartmentalisation of carboplatin in the brain over many hours should ensure that a clinically significant tumour cell kill is achieved whilst negligible plasma levels of carboplatin are maintained. Indeed, through the use of an implanted catheter system, repeated bolus infusions of carboplatin should facilitate maintenance of a relatively constant carboplatin concentration within the peritumoural tissue for a predetermined period of time.

Having identified that carboplatin remains in the brain for at least 24 hours, we examined the relationships between carboplatin concentration and duration of exposure on the tumour cell kill achieved. Unsurprisingly, as the carboplatin concentration and exposure duration were increased, the proportion of tumour cells that were killed increased. This effect appeared to plateau with carboplatin concentrations of 0.18 mg/ml and 0.24 mg/ml at exposure durations of 96 hours and 72 hours respectively. Although it is difficult to accurately simulate the effects of cytotoxic agents in vitro, particularly due to the lack of tumour cell heterogeneity, which is a feature of GBM, these results imply that using appropriate carboplatin concentrations, a maximal tumour cell kill could be achieved by maintaining a therapeutic carboplatin concentration in peritumoural brain for 3 to 4 days. In view of the tissue half-life of carboplatin that we have demonstrated in the brain and with the use of an implanted catheter system it should be feasible to effectively administer carboplatin to peritumoural brain for these periods of time in clinical practice. From a practical perspective, this approach would be similar to the phase III clinical trial of the immunotoxin IL13-PE38QQR, which was administered through 2 to 4 catheters for 96 hours, to patients with recurrent glioblastoma.

In an attempt to determine whether carboplatin administered by CED was associated with significant toxicity in rats, we undertook a dose-escalation study. This study demonstrated no clinical or histological evidence of toxicity at concentrations of up to 0.9 mg/ml. This result supports previous studies in rats and primates that would suggest that carboplatin can be safely administered into the brain at a potentially efficacious dose. Specifically, Degen et al. undertook single infusions of carboplatin into the brainstem of rats at concentrations as high as 1 mg/ml without histological evidence of tissue damage, and Strege et al. undertook one-month long infusions into the brainstem of primates at a dose of 0.075 mg/kg with minimal clinical evidence of toxicity, manifesting as slight slowing of the animal's movements. In view of our in vitro results demonstrating an optimal tumour cell kill following prolonged infusions over several days; our toxicity study is limited by the fact that single infusions were performed. Ideally, we would have liked to have replicated infusions of 0.24 mg/ml over 72 hours and 0.18 mg/ml over 96 hours. However, whereas a clinical trial could employ short CED-based bolus infusions at intervals to achieve a steady-state concentration in the brain, due to the small size of the rat brain, continuous, low-rate infusions would need to be performed. These infusions would most likely have led to a toxic build-up of carboplatin in the brain, particularly around the catheter tip, at potentially much higher concentrations than the infused concentration. Consequently, undertaking continuous infusions into rat brain could have led to misrepresentative toxicity data and therefore they were not performed.

A key consideration in the application of CED in clinical trials is the need to visualise infusate distribution to ensure that adequate drug distribution is achieved through the intended target volume. A simple strategy that has previously been employed in clinical practice is the coinfusion of an MR contrast agent such as gadolinium-DTPA. As gadolinium is detectable by LA-ICP-MS, it was possible to evaluate the differential distribution properties of gadolinium-DTPA and carboplatin in the brain of a large animal model in which in vivo T1-weighted MR imaging could be performed. It is perhaps unsurprising that more widespread distribution of gadolinium was demonstrated with LA-ICP-MS compared to T1-weighted MRI, in view of the greater sensitivity of the former technique. Nevertheless, from the perspective of undertaking a clinical trial, it was encouraging that the area of contrast-enhancement on T1-weighted MR imaging approximately matched carboplatin distribution determined by LA-ICP-MS. Although, CED should lead to homogenous infusate distribution this was not the case with gadolinium-DTPA distribution visualised by LA-ICP-MS, presumably due to the ability of gadolinium-DTPA to diffuse through the brain extracellular space. Consequently, for future trials involving the administration of gadolinium-DTPA, the effect of modulating the infused concentration on the visualisation of contrast-enhancement by MRI would be invaluable.

In conclusion, this study provides experimental evidence that carboplatin can be efficiently administered into the brain by CED. In addition, due to its slow clearance from the brain and toxicity to glioblastoma cells at concentrations that are not toxic to normal brain, carboplatin administration by CED into peritumoural brain represents a promising therapeutic approach to treating patients with recurrent glioblastoma multiforme.

### EXAMPLE 3

The toxicity of carboplatin was analysed by measuring the levels of synaptophysin in the brain. A reduction in synaptophysin is indicative of toxicity and was observed at 0.72 mg/ml carboplatin but not at 0.36 mg/ml (FIG. 6).

Brain tissue homogenates were prepared from dissected samples of unfixed frozen hemispheres from rats at 72 hours after infusion of either artificial CSF (control) or carboplatin at concentrations of 0.36 and 0.72 mg/ml. Tissue samples incorporating the overlying cerebral cortex (approximately 200 mg) were dissected and homogenised for 75 s using a Precellys 24 automated tissue homogeniser (Stretton Scientific) with 2.3 mm silica beads (Biospec) in 1% SDS, 10 mM tris base (pH 6.0), 0.1 mM sodium chloride, and the protease inhibitors aprotinin (1 μg/ml; Sigma) and PMSF (10 μM; Sigma). The resultant crude tissue homogenates were centrifuged at 13,000 rpm for 15 min at 4° C., and the supernatants aliquoted and stored at −80° C.

Ninety-six-well plates (Nunc Maxisorp) were coated with primary rabbit anti-synaptophysin polyclonal antibody (Abeam) at a concentration of 1 μg/ml, and incubated overnight at 4° C. After 5 washes with wash buffer, non-specific binding was blocked with the addition of 1% BSA/PBS for 2 hours. After 5 washes with wash buffer, serial dilutions of recombinant synaptophysin or the supernatant of crude homogenates were added to wells in triplicate, and incubated at room temperature for 2 hours. After 5 further washes with wash buffer, secondary antibody (mouse monoclonal anti-synaptophysin, Santa Cruz, Calif., US) was used at 1:1000 in 1% BSA/PBS, and incubated for 2 hours. Tertiary antibody (HRP-labelled anti-mouse antibody, Sigma) was added to wells at 1:200 after 5 washes, and incubated for 30 minutes in the dark. Peroxidase substrate (R&D Systems) was added to wells for 5 min, then the reaction stopped with STOP solution (R&D Systems). Plates were read in a microplate reader at 405 nm (BMG Labtech).

### EXAMPLE 4

### History

A 5 year-old boy presented with a 1 month history of unsteady gait, intermittent diplopia and swallowing difficulty. Contrast magnetic resonance imaging (MRI) revealed a large mass lesion expanding the pons and midbrain with patchy areas of enhancement extending superiorly along the right cerebral peduncle and consistent with a diagnosis of diffuse intrinsic pontine glioma. He was commenced on oral dexamethasone and then treated with a 6 week course of radiotherapy, resulting in stabilisation of his neurological condition for approximately 3 months. After this time he developed progressive left sided weakness and dysphagia requiring increases in his dexamethasone dose.

At 9 months after diagnosis, the patient's clinical status deteriorated as he developed dysphasia, progressively worsening trismus, dysphagia and lethargy. Following review by the paediatric neuro-oncology multidisciplinary team and approval from our Institutional Review Board, a decision was made to proceed with convection-enhanced delivery of carboplatin.

### Pre-Operative Planning

MR imaging under general anaesthetic was undertaken to facilitate pre-operative stereotactic planning (field strength 3 T, Philips Achieva TX, Philips Healthcare, The Netherlands) one week prior to surgery. This imaging confirmed a significant increase in tumour size with extension of the tumour along the left cerebral peduncle and patchy areas of necosis. Using an in-house modification to Neuro|Inspire™ Stereotactic Planning Software (Renishaw Plc, Wotton-under-Edge, Gloucs., UK) the total tumour volume was calculated as 43.6 ml, including 6.8 ml of necrotic areas. A left transfrontal trajectory for catheter implantation was planned (FIGS. 7a-d) facilitating the in-house manufacture of a bespoke catheter with a winged hub (FIG. 8a). The catheter was manufactured from polyether ether ketone (PEEK) with an outer diameter (OD) of 0.6 mm, which was bonded onto a fused silica cannula with a laser-cut tip (OD 0.23 mm). The catheter was designed to be implanted through a 1 mm OD carbothane guide-tube.

### Surgical Procedure

On the day of surgery the patient was anaesthetised and placed in a Leksell frame. A pre-operative CT angiogram was performed and co-registered with the post-contrast T1 weighted planning MRI scan to facilitate output of stereotactic co-ordinates to a Neuro|Mate® neurosurgical robot (Renishaw) (FIG. 8b). A 3 cm left frontal curvilinear scalp incision was made and the periosteum retracted. The robot was driven to the entry position on the skull, and using custom-made hand drills, a multi-featured burr hole made into which the guide tube hub would push fit. The dura was pierced and a 1 mm guide rod inserted to a point 24 mm proximal to the target within the tumour. The guide tube was then implanted on a 0.6 mm guide rod to maintain trajectory. The catheter was tunnelled out through a separate stab incision in the scalp and connected to a custom-made in-line gas and bacterial filter. The catheter was attached to an infusion pump (B Braun, Melsungen, Germany) and primed with artificial cerebrospinal fluid (Torbay Pharmaceutical Manufacturing Unit, Torbay, UK). The fused silica catheter was then implanted via the guide-tube with 3 mm of fused silica retained within the guide tube and 24 mm extending beyond the guide-tube tip, thus creating a “recessed-step” within the distal guide-tube (FIG. 8c). The winged hub of the catheter was turned 90° at the skull and secured with 5 mm titanium screws. The distance from skull surface to catheter tip was 105 mm. The skin incision was closed in layers and the externalised catheter tubing secured in a loop on the scalp (FIG. 8d).

### Infusions of Carboplatin

Whilst under general anaesthesia the child was transferred to the 3 T MRI scanner. The externalised catheter was attached to a 6 m extension line to allow infusions to be performed from a syringe driver (B Braun) outside of the scan room. Infusions of carboplatin diluted in artificial cerebrospinal fluid to a concentration of 0.09 mg/ml were commenced using the following infusion regime:

0.5 μl/min for 10 minutes, 1 μl/min for 5 minutes, 2.5 μl/min for 5 minutes, 5 μl/min for 5 minutes, 7.5 μl/min until completion.

Serial real-time T2-weighted MRI scans were performed in order to allow areas of hyperintense signal change to be used as a proxy measure for drug distribution. The volume of distribution was estimated to be 2.06 ml after infusion of 0.52 ml of carboplatin, resulting in an approximate volume of infusion (Vi) to distribution (Vd) ratio of 4. Based on this Vi:Vd ratio the volume of infusion required to fill the full tumour volume (excluding necrotic areas) was estimated to be approximately 9 ml. Once the Vi:Vd ratio was established the child was recovered from anaesthesia and monitored in a high dependency area until completion of the infusion. The total infusion time was 20 hours. Infusions of carboplatin were performed on 3 consecutive days in order to maintain exposure to the cytotoxic chemotherapy for at least 72 hours. A total volume of 26.6 ml was infused into the tumour over 3 days.

After a 4 day break in treatment, the fused silica catheter and external filter were exchanged for a new system under a short general anaesthetic. Infusions of carboplatin were re-commenced at a concentration of 0.18 mg/ml at a maximum infusion rate of 10 μl/min, and repeated on 2 consecutive days. Infusion volumes, times and maximum flow rates are shown in Table 1. On completion of the final infusion, a T2-weighted MRI scan was performed in order to allow volumetric analysis of signal change as a proxy measure of the final infusate distribution. The volume of T2 signal change was measured as 35.1 ml, suggesting drug distribution throughout approximately 95% of the targeted tumour volume (FIG. 9a & b). There was no evidence of reflux along the guide-tube on T2-weighted MR imaging.

TABLE 1
Schedule of infusion

| | Carboplatin | Maximum | Infusion | |
|---|---|---|---|---|
| | concentration | infusion rate | volume | Infusion time |
| Day | (mg/ml) | (μl/min) | (ml) | (Hours) |
| 1 | 0.09 | 7.5 | 8.73 | 20 |
| 2 | 0.09 | 7.5 | 8.98 | 20 |
| 3 | 0.09 | 7.5 | 8.92 | 20 |
| 8 | 0.18 | 7.5 | 8.96 | 20 |
| 9 | 0.18 | 10 | 14.2 | 24 |

The microcatheter implantation procedure and infusions of carboplatin were well tolerated, and not associated with any reduction in conscious level. During the infusions, the patient experienced a transient worsening in neurological status with worsening of his trismus and swallowing difficulty. These changes were reversible on cessation of the third infusion, and his neurological status returned to baseline over the following 24 hours. The catheter was removed on day 12 and replaced with a stylet. The guide-tube remained in situ to facilitate further catheter implantations without the need for application of a stereotactic frame or robot-guidance. The patient was discharged home on day 14.

### Results

### Clinical and Radiological Follow-Up

The patient was re-admitted one month after completion of the infusions for clinical review and MR imaging. He demonstrated increased alertness and interaction with his family and showed some improvement in left arm function. He had also tolerated a reduction in steroid dosage from 2 mg dexamethasone twice daily to 0.8 mg twice daily, something which was not possible prior to treatment. However, there was evidence of worsening axial stability and he continued to suffer with intermittent trismus, dysphasia and dysphagia. Follow-up T2-weighted MR imaging revealed areas of increased signal change throughout the volume of infusate distribution, suggestive of the early stages of tumour necrosis (FIG. 10a-f). However, post-contrast T1-weighted imaging confirmed tumour progression in the inferior and anterior regions of the tumour. The areas of tumour progression were outside of the volume of T2 signal change visualised on completion of the infusions.

Unfortunately the child died two months following completion of treatment after suffering a rapid deterioration in neurological status with reduced conscious level, and developing signs and symptoms suggestive of aspiration pneumonia.

### Discussion

In this case we were able to safely and accurately deliver a microcatheter with a 0.23 mm outer diameter to an intra-tumoral target at a depth of 105 mm from the skull surface. By employing a stable robot-guided platform for catheter implantation our intention was to minimise tissue trauma on guide-tube and catheter implantation, thus reducing the risk of reflux. The novel recessed-step feature (manuscript in preparation) may also have contributed to the achievement of reflux-free infusions by creating an effective seal at the interface between the guide-tube tip and surrounding brain. We believe that the combination of a robot-guided implantation method and recessed-step catheter design allowed us to achieve high volume, high flow rate infusions without reflux.

The use of T2-weighted MR imaging for volume of distribution analysis has been described in previous clinical studies, and it has been reported that the volume of T2 signal change significantly underestimates the true volume of drug distribution. In this case we used serial real-time T2-weighted MRI scans to estimate the Vi:Vd ratio thus allowing us to estimate the total volume of infusion required to achieve drug distribution throughout the target tumour volume. On completion of the final infusion of carboplatin, the volume of T2 signal change was measured as 35.1 ml, representing 95% of the targeted tumour volume. The areas of tumour progression on follow-up imaging were outside of this volume suggesting inadequate drug delivery to the peripheral areas of tumour. We would therefore advocate the use of multiple catheter trajectories for the future treatment of advanced and very large brainstem tumours.

This case demonstrates the feasibility of accurately and safely delivering very small diameter catheters to deep targets within the brainstem using a robot-guided catheter implantation procedure. Large volume infusions were well tolerated at flow rates as high as 10 μl/min, without evidence of reflux. Based on T2-weighted MR imaging, infusate distribution was achieved throughout the majority of the tumour volume, and we are hopeful that this treatment strategy could favourably impact the prognosis of patients with smaller tumours treated at earlier stages of the disease process.

1. A method of treating a glioma comprising administering to a patient in need thereof a composition comprising a chemotherapy agent and artificial cerebrospinal fluid via convection enhanced delivery.

2. The method according to claim 1, wherein the chemotherapy agent is delivered to white matter via convection enhanced delivery.

3. The method according to claim 1, wherein the white matter is within 30 mm of a glioma or of a site from which a glioma has been resected.

4. The method according to claim 1, wherein the chemotherapy agent is administered at a concentration of from 0.01 mg/ml to 0.30 mg/ml.

5. The method according to claim 1, wherein the chemotherapy agent is administered for a period of 4 to 24 hours.

6. The method according to claim 1, wherein the chemotherapy agent is administered on at least two consecutive days.

7. The method according to claim 1, wherein the chemotherapy agent is administered at a flow rate of at least 6 μl/min.

8. The method according to claim 1, further comprising resecting part or all of a glioma.

9. The method according to claim 1, further comprising implanting a distal end of two or more catheters in white matter within 5 to 30 mm of a glioma or of a site from which a glioma has been resected.
